(19) United States
(12) Patent Application Publication
(10) Pub. No.: US 2024/0302387 A1
(43) Pub. Date: Sep. 12, 2024

(54) HIGHLY FLUORINATED COMPOUNDS FOR 19F MRI-BASED THERMOMETRY

(71) Applicant: Regents of the University of Minnesota, Minneapolis, MN (US)

(72) Inventors: William C.K. Pomerantz, Minneapolis, MN (US); Jiaqian Li, Minneapolis, MN (US)

(73) Assignee: Regents of the University of Minnesota, Minneapolis, MN (US)

(21) Appl. No.: 18/403,891

(22) Filed: Jan. 4, 2024

Related U.S. Application Data
(60) Provisional application No. 63/437,255, filed on Jan. 5, 2023.

(51) Int. Cl. G01N 33/84; C07C 317/14; C07C 321/28; C07C 321/30

(52) U.S. Cl. CPC G01N 33/84; C07C 317/14; C07C 321/28; C07C 321/30

(57) ABSTRACT

The invention provides compounds of Formula I useful for the measurement of temperature by ¹⁹F magnetic resonance imaging.

### CROSS REFERENCE TO RELATED APPLICATIONS

This non-provisional application claims the benefit of priority to U.S. provisional Application 63/437,255, filed 5 Jan. 2023, which is incorporated by reference in entirety.

### FIELD OF THE INVENTION

The invention relates generally to compounds for the measurement of temperature by 19F MRI-based thermometry.

### BACKGROUND OF THE INVENTION

Non-invasive measurement of temperature is vital for informing on local tissue temperatures. These measurements are used for evaluating disease pathology and medical interventions such as high intensity focused ultrasound, low temperature hyperthermia, hypothermia, radiofrequency ablation, and thermal ablation (Rieke V, et al (2008) J Magn Reson Imaging 27(2):376-90). Additionally, in vivo oximetry measurements are also sensitive to temperature fluctuations, requiring accurate temperature determination. Magnetic resonance imaging (MRI) is one such approach that is used in the clinic. The gold standard thermometry measurement is based on changes in the proton-resonance frequency (PRF) of water with the highest responsiveness in most tissues of ˜10×10⁻³ ppm/° C. (Hindman J C. (1966) J Chem Phys 44:4582-4592; McDannold N. (2005) Int J Hyperthermia 21:533-546; Rieke V, et al (2008) J Magn Reson Imaging 27(3):673-7) or absolute temperature measurements, however reference frequencies are required. PRF temperature measurements are also susceptible to error from magnetic field drift and have low sensitivity in fatty tissue (Rieke et al (2008) Jour. Mag. Res. Imaging 27:376-390). Such limitations have led to the development of heteronuclear approaches including using ²³Na, ¹²⁹Xe, and ¹⁹F (Schilling, F. et al. (2010) ChemPhysChem 11:3529-3533).

While fluorine-based magnetic resonance applications have been mainly limited to preclinical studies due to current limitations in the availability of ¹⁹F MRI systems for clinical use, there are several advantages of ¹⁹F-magenetic resonance that make this an attractive strategy for further development. Given it similar gyromagnetic ratio to ¹H, ¹⁹F is the second most sensitive stable NMR active nucleus, with 83% signal sensitivity relative to ¹H. In addition, the absence of naturally occurring mobile fluorine, provides a background-free spectrum that can be used in combination with ¹H MRI for anatomical imaging. Organofluorine temperature sensors have previously been developed based on changes in nuclear relaxation or chemical shift changes (Prinz, C., et al (2019) Magn. Reason. Mater Phy. 32:51-61; Thorarindsdottir, A. F., et al (2017) Chem. Sci. 8:2448-2456). Perfluorocarbon liquids are particularly attractive, due to their ability to be formulated into nanoemulsions or encapsulated into nanoparticles to help overcome the inherently low sensitivity of magnetic resonance measurements for in vivo cell tracking studies, oximetry, and thermometry.

The perfluorocarbon liquid, perfluorotributylamine (PFTBA) was the first organofluorine compound to be used for in vivo thermometry with a temperature responsiveness of ˜9×10⁻³ ppm/° C. approaching the PRF responsiveness and remains one of the most responsive ¹⁹F sensors.^{[11]} Given the temperature dependence of fluorocarbon-based oximetry agents, such as hexafluorobenzene which is based on T1 relaxation (Katki, H., et al (1996) NAMR in Biomedicine 9(3):135-139). PFTBA can be used in combination for temperature calibration (R. P. Mason, R. P. (1994) Artificial Cells, Blood Substitutes, and Biotechnology 22(4):1141-1153). However, several potential difficulties are encountered when using PFTBA including multiple resonances with similar frequencies, which may lead to chemical shift artifacts and a responsiveness below the PRF (Parsa, J., et al (2021) Journal of Magnetic Resonance 325:106946). Additionally, PFTBA is a potent greenhouse gas (Hong, A. C., et al (2013) Geophys. Res. Lett., VOL. 40, 6010-6015) in addition to concerns of environmental persistence due to the perfluorinated alkyl groups. Certain organofluorine temperature sensors improve upon the PFTBA temperature responsiveness and PRF by almost 2-fold ((Lee, A. L., et al (2022) Analytical Chemistry 94(9):3782-3790; Lee, A. L., et al (2021) Langmuir 37(17):5222-5231). Known organofluorine temperature sensors have too many fluorine resonances of similar chemical shift which could lead to chemical shift artifacts for ¹⁹F MRI imaging. In addition, the long polyfluorinated tails raise concerns over environmental persistence. Finally, while PFTBA is a liquid, and is readily formulated in mesoporous silica nanoparticles, the known organofluorine temperature sensors are solids making this process significantly more challenging.

### SUMMARY

The invention is generally directed to a compound of Formula I:

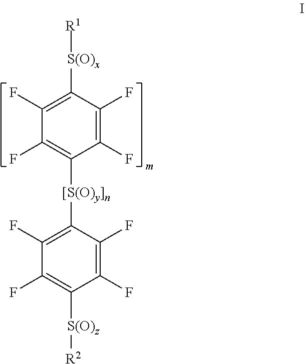

or a pharmaceutically acceptable salt, stereoisomer, mixture of stereoisomers, tautomer, or isotopic analog thereof, wherein:
R¹ and R² are independently selected from —(C₃-C₆ alkyldiyl)-(CF₂)ₚ—CF₃, where p is 1 to 10;
x, y, and z are independently selected from 0, 1, and 2; and
m and n are independently selected from 0 and 1, where if n is 1, then m is 1.

Another aspect of the invention is a method of preparing Formula I compounds.

Another aspect of the invention is a nanoparticle formulation comprising a compound of Formula I and a nanoparticle.

Another aspect of the invention is a method for measurement of temperature in a biological sample comprising detecting a Formula I compound in a biological sample by ¹⁹F magnetic resonance imaging at two or more temperatures.

### DESCRIPTION OF THE DRAWINGS

FIG. 1 shows the structure and spectrum of DD-1 in THF-d₈ at 27° C. Fluorine groups matched the 2 resonances determining the temperature responsiveness of DD-1.

FIG. 2A shows the selected regions of the ¹⁹F NMR spectra of CF₂ groups of DD-1. Resonances move downfield as temperature increases from 27 to 47° C.

FIG. 2B shows the selected regions of the ¹⁹F NMR spectra of Ar—F group of DD-1. Resonances move upfield as temperature increases from 27 to 47° C.

FIG. 3 shows the temperature response of DD-1 in THF-d₈ with 15.98±0.05×10⁻³ ppm ° C.-1 (n=3, mean±SD).

FIG. 4 shows the temperature response of DD-1 in phosphate buffer with 17.57±0.05×10⁻³ ppm ° C.⁻¹ (n=3, mean±SD).

FIG. 5 shows photochemical degradation kinetic plots of DD-1, DD-3, and DD-5 at 10 μM with photolysis (filled) and dark controls (empty) in (A) the mercury vapor lamp. Experiments were in 10 mM pH 7 phosphate buffer and contained residual DMSO from the stock solution. First order rate constants and 95% confidence intervals shown for each compound. Error bars indicate the standard deviation of three samples. Inverted triangles refer to DD-1, regular triangle refers to DD-3, and the squares refer to DD-5.

FIG. 6 shows photochemical degradation kinetic plots of DD-1, DD-3, and DD-5 at 10 μM with photolysis (filled) and dark controls (empty) in the solar simulator. Experiments were in 10 mM pH 7 phosphate buffer and contained residual DMSO from the stock solution. First order rate constants and 95% confidence intervals shown for each compound. Error bars indicate the standard deviation of three samples. Inverted triangles refer to DD-1, regular triangle refers to DD-3, and the squares refer to DD-5.

FIG. 7 shows ¹⁹F-NMR spectra of DD-1 showing chemical shifts of fluorinated groups in the parent compound and reaction products. Top: Selected spectral regions of untreated DD-1. Middle: Selected spectral regions of DD-1 following partial degradation by the solar simulator. Bottom: Selected spectral regions of DD-1 following partial degradation by ozone. Fluoride (F⁻) produced from degradation reactions is indicated for clarity. Spectra in the left, middle, and right regions are scaled differently for visibility.

FIG. 8A shows the temperature response of aryl ¹⁹F resonances in the spectra (aromatic region) of the non-sulfone containing temperature sensor DD-1 where aromatic resonance(s) move upfield with the increase of temperature.

FIG. 8B shows the temperature response of aryl ¹⁹F resonances in the spectra (aromatic region) of the sulfone containing temperature sensor DD-2 where aromatic resonance(s) move downfield with the increase of temperature.

FIG. 8C shows the temperature response of aryl ¹⁹F resonances in the spectra (aromatic region) of the non-sulfone containing temperature sensor DD-3 where aromatic resonance(s) move upfield with the increase of temperature.

FIG. 8D shows the temperature response of aryl ¹⁹F resonances in the spectra (aromatic region) of the sulfone containing temperature sensor DD-4 where aromatic resonance(s) move downfield with the increase of temperature.

FIG. 8E shows the temperature response of aryl ¹⁹F resonances in the spectra (aromatic region) of the non-sulfone containing temperature sensor DD-5 where aromatic resonance(s) move upfield with the increase of temperature.

FIG. 8F shows the temperature response of aryl ¹⁹F resonances in the spectra (aromatic region) of the sulfone containing temperature sensor DD-6 where aromatic resonance(s) move downfield with the increase of temperature.

FIG. 9A shows the ¹⁹F NMR spectrum of DD-1 in phosphate buffer pH=6.9.

FIG. 9B shows a table of normalized signal intensity of individual resonance at different concentrations of DD-1 (top). All signal intensities of resonances of 100 μM DD-1 were set to 1. At the bottom left is a normalized intensity chart of 3 individual resonances at different concentrations of DD-1, excluding 250 μM. At the bottom right is a normalized intensity chart of 3 individual resonances at different concentrations of DD-1, including 250 μM (micromolar). The R² of the CF₃ (−87 ppm) resonance line is only 0.9776.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the invention as defined by the claims.

One skilled in the art will recognize many methods and materials similar or equivalent to those described herein, which could be used in the practice of the present invention. The invention is in no way limited to the methods and materials described.

### Definitions

The term “alkyl” refers to a straight (linear) or branched, saturated, aliphatic radical having the number of carbon atoms indicated. Alkyl can include any number of carbons, for example from one to twelve. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, 1-octyl, and the like. Alkyl groups can be substituted or unsubstituted. “Substituted alkyl” groups can be substituted with one or more groups selected from halo, hydroxy, amino, oxo (═O), alkylamino, amido, acyl, nitro, cyano, and alkoxy.

The term “alkyldiyl” refers to a divalent alkyl radical. Examples of alkyldiyl groups include, but are not limited to, methylene (—CH₂—), ethylene (—CH₂CH₂—), propylene (—CH₂CH₂CH₂—), and the like. An alkyldiyl group may also be referred to as an “alkylene” group.

### Design and Synthesis of Organofluorine Temperature Sensors:

To improve the properties of phase behavior and thermal responsiveness, highly fluorinated small molecules (organofluorines) were designed through variation in the aromatic core, fluorinated tail length and heteroatom oxidation state (DD1-DD7, Table 1a). All temperature sensors maintained structural symmetry for increasing the number of magnetically equivalent fluorine atoms for high signal sensitivity while reducing the overall number of fluorine resonances which could lead to potential artifacts in ¹⁹F magnetic resonance applications. While comparator compound CC-2 has a perfluorobiphenyl core, a similarly responsive comparator compound CC-3 has a more simplified perfluorophenyl core (Table 1b). Inclusion of these aryl fluorine groups was important as the thermal responsiveness of the respective ¹⁹F resonances were essential for maintaining a high temperature responsiveness. A perfluorophenyl sulfide and a perfluorophenyl sulfone core were explored to evaluate if these functional groups would lead to additional thermal responsiveness or alter the phase behavior of the final compound through introduction of two additional rotatable bonds. A shorter alkyl chain containing a perfluoroethyl group was predicted to slightly improve the thermal responsiveness while reducing the number of fluorine atoms from the highly fluorinated —(CF₂)₇—CF₃ alkyl chains of comparator compounds CC-2 and CC-3. Such an alkyl chain was anticipated to reduce potential chemical shift artifacts and reduce environmental persistence. The —S—(CH₂)₃—CF₂—CF₃ group found in DD1-DD6 maintains the CF₂ group adjacent to a CH₂ which was the second most responsive fluorine group in CC-2 and CC-3. Sulfide oxidation states were modified in DD-2, DD-4, and DD-6 to evaluate the heteroatom effects on thermal responsiveness.

All seven organofluorine temperature sensors of Table 1a were synthesized under the optimized reaction conditions for enabling facile synthesis in the following General Scheme and Examples. Specifically, temperature sensors DD1-DD7 were prepared through an S_{N}Ar reaction. Compounds DD-2 and DD-4 required one extra oxidation of DD-1 and DD-3 to provide the respective sulfones. Yields of the S_{N}Ar reactions using thiol, HS—(CF₂)₇—CF₃, were low 2100 to 24%, while the yields with HS—CH₂CH₂CH₂CF₂CF₃ fell in the range of 5300 to 78%. In the case of DD-2 and DD4-DD7 the sulfur oxidation state or incorporation within the aromatic core did not lead to altered phase behavior at room temperature resulting in white solids. However, in the case of DD-1 and DD-3 which possess the shorter fluorinated alkyl chains and either a perfluorophenyl or perfluorobiphenyl group respectively, a room temperature liquid phase was obtained.

TABLE la
Exemplary DD Formula I compounds with phase data

| DD | | | | |
|---|---|---|---|---|
| No. | Structure | Name | MW | Phase |
| DD-1 | 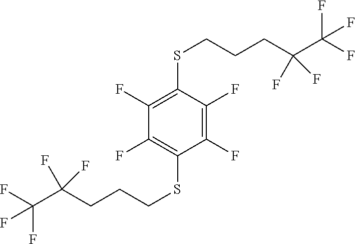 [SMILES: Fc1c(F)c(SCCCC(F)(F)C(F)(F)F)c(F)c(F)c1SCCCC(F)(F)C(F)(F)F] | (perfluoro-1,4- phenylene)bis((4,4,5,5,5- pentafluoropentyl)sulfane) | 534.4 | liquid |
| DD-2 | 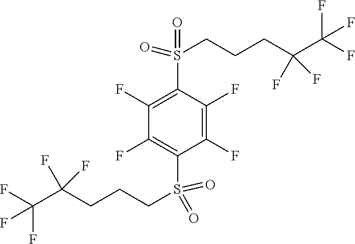 [SMILES: O=S(=O)(CCCC(F)(F)C(F)(F)F)c1c(F)c(F)c(S(=O)(=O)CCCC(F)(F)C(F)(F)F)c(F)c1F] | 1,2,4,5-tetrafluoro-3,6- bis((4,4,5,5,5- pentafluoropentyl)sulfonyl) benzene | 598.4 | solid |
| DD-3 | 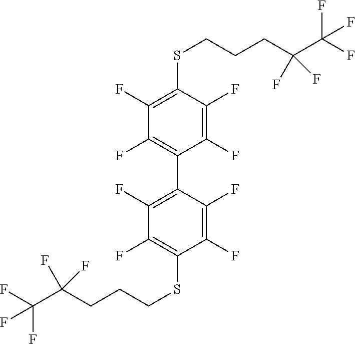 [SMILES: Fc1c(F)c(-c2c(F)c(F)c(SCCCC(F)(F)C(F)(F)F)c(F)c2F)c(F)c(F)c1SCCCC(F)(F)C(F)(F)F] | (perfluoro-[1,1′-biphenyl]- 4,4′-diyl)bis((4,4,5,5,5- pentafluoropentyl)sulfane) | 682.4 | liquid |
| DD-4 | 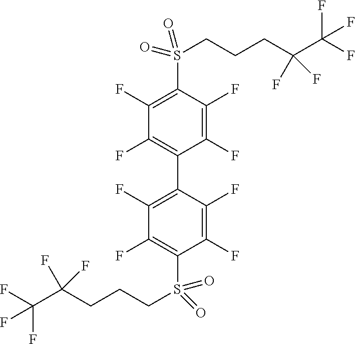 [SMILES: O=S(=O)(CCCC(F)(F)C(F)(F)F)c1c(F)c(F)c(-c2c(F)c(F)c(S(=O)(=O)CCCC(F)(F)C(F)(F)F)c(F)c2F)c(F)c1F] | 2,2′,3,3′,5,5′,6,6′- octafluoro-4,4′- bis((4,4,5,5,5- pentafluoropentyl) sulfonyl)-1,1′-biphenyl | 746.4 | solid |
| DD-5 | 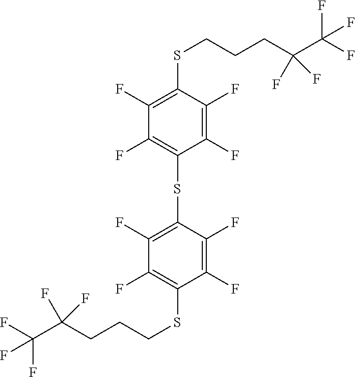 [SMILES: Fc1c(F)c(Sc2c(F)c(F)c(SCCCC(F)(F)C(F)(F)F)c(F)c2F)c(F)c(F)c1SCCCC(F)(F)C(F)(F)F] | bis(2,3,5,6-tetrafluoro-4- ((4,4,5,5,5- pentafluoropentyl)thio) phenyl)sulfane | 714.5 | solid |
| DD-6 | 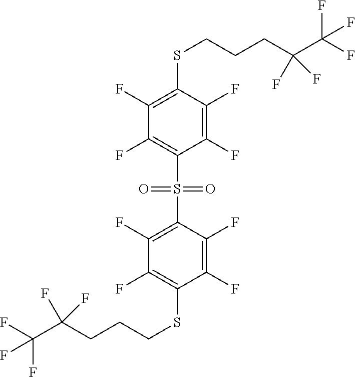 [SMILES: O=S(=O)(c1c(F)c(F)c(SCCCC(F)(F)C(F)(F)F)c(F)c1F)c1c(F)c(F)c(SCCCC(F)(F)C(F)(F)F)c(F)c1F] | (sulfonylbis(2,3,5,6- tetrafluoro-4,1- phenylene))bis((4,4,5,5,5- pentafluoropentyl)sulfane) | 746.5 | solid |
| DD-7 | 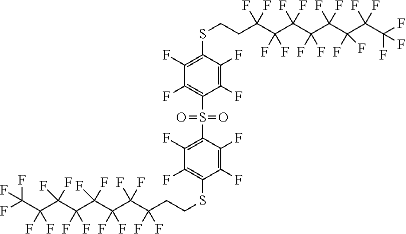 [SMILES: O=S(=O)(c1c(F)c(F)c(SCCC(F)(F)C(F)(F)C(F)(F)C(F)(F)C(F)(F)C(F)(F)C(F)(F)C(F)(F)F)c(F)c1F)c1c(F)c(F)c(SCCC(F)(F)C(F)(F)C(F)(F)C(F)(F)C(F)(F)C(F)(F)C(F)(F)C(F)(F)F)c(F)c1F] | (sulfonylbis(2,3,5,6- tetrafluoro-4, 1- phenylene))bis((3,3,4,4,5,5, 6,6,7,7,8,8,9,9,10,10,10- heptadecafluorodecyl) sulfane) | 1318.5 | solid |

TABLE 1b
Comparator compounds (CC) with phase data

| CC No. | Structure | Name | MW | Phase |
|---|---|---|---|---|
| CC-1 | N(CF₂CF₂CF₂CF₃)₃ | perfluorotributylamine | 671.1 | liquid |
| | | (PFTBA) | | |
| CC-2 | 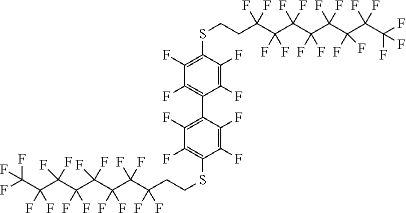 [SMILES: Fc1c(F)c(-c2c(F)c(F)c(SCCC(F)(F)C(F)(F)C(F)(F)C(F)(F)C(F)(F)C(F)(F)C(F)(F)C(F)(F)F)c(F)c2F)c(F)c(F)c1SCCC(F)(F)C(F)(F)C(F)(F)C(F)(F)C(F)(F)C(F)(F)C(F)(F)C(F)(F)F] | (perfluoro-[1,1′- biphenyl]-4,4′- diyl)bis((3,3,4,4,5,5,6,6, 7,7,8,8,9,9,10,10,10- heptadecafluorodecyl) sulfane) | 1254.5 | solid |
| CC-3 | 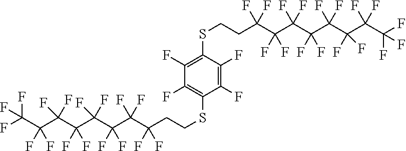 [SMILES: Fc1c(F)c(SCCC(F)(F)C(F)(F)C(F)(F)C(F)(F)C(F)(F)C(F)(F)C(F)(F)C(F)(F)F)c(F)c(F)c1SCCC(F)(F)C(F)(F)C(F)(F)C(F)(F)C(F)(F)C(F)(F)C(F)(F)C(F)(F)F] | (perfluoro-1,4- phenylene)bis((3,3,4,4, 5,5,6,6,7,7,8,8,9,9,10,1 0,10- heptadecafluorodecyl) sulfane) | 1106.4 | solid |

### Results

Structure-property Relationship Studies of Fluorinated Temperature Sensors:

The temperature responsiveness of the Table 1a compounds was investigated along with comparator compounds of Table 1b; PFTBA CC-1, CC-2, and CC-3. Analyses were conducted in THF, a solvent where all fluorinated molecules were soluble. Table 2 shows the responsiveness for DD1-DD7, and the comparator compounds PFTBA, CC-2, and CC-3.

Temperature responsiveness was determined based on linear plotting of the chemical shift difference (ΔΔδ) of the resonance pair at difference temperature (T). Resonances moving to the most upfield resonance and moving downfield with the largest change as a function of temperature were chosen as resonance pairs. The slope of the linear plot was used to determine the temperature responsiveness.

Thermal responsiveness is only reported for the two most responsive pairs of fluorine resonances, which in most cases remain the CF₂ group adjacent to a methylene CH₂ group, and an aryl fluorine group. Under these conditions, all compounds led to a significant increase in responsiveness of 1900 (DD-2) to up to 7500 (DD-3) relative to PFTBA with a responsiveness of 9.45×10⁻³ ppm/° C. under these conditions.

TABLE 2
Responsiveness data of DD Formula
I compounds and PFTBA

| | | Responsiveness |
|---|---|---|
| | | in aqueous |
| | Responsiveness | phosphate |
| DD No. | in THF-d₈ | buffer |
| DD-1 | 16.0 | 17.6 |
| DD-2 | 12.7 | |
| DD-3 | 16.5 | 17.0 |
| DD-4 | 12.9 | |
| DD-5 | 16.4 | |
| DD-6 | 11.5 | |
| DD-7 | 12.8 | |
| CC-1 | 9.45 | 9.62 |
| PFTBA | | |
| CC-2 | 14.5 | |
| CC-3 | 14.2 | |

The effect on responsiveness revealed a significant structure-property relationship. First the effect of short alkyl chains with fewer ¹⁹F atoms can be analyzed. Comparing matched sensor pairs with the same aromatic core and heteroatom oxidation state, sensors with side chains of shorter length and fewer ¹⁹F atoms were at least 14% more responsive with the exception of DD-6 and DD-7 which showed a close responsiveness. Therefore, the short alkyl chain with fewer ¹⁹F atoms remains the most beneficial for responsiveness.

The effect of different aromatic cores on responsiveness was next evaluated. DD-1, DD-3, DD-5 and DD-6 all have the same —CH₂CH₂CH₂CF₂CF₃ fluorinated alkyl chain. In this case, DD-1, DD-3 and DD-5 have comparable responsiveness at ˜16×10⁻³ ppm/° C., while DD-6 containing the perfluorobiphenylsulfone core was significantly less responsive (11.53×10⁻³ ppm/° C.) than the other three sensors. These results indicated that of the four proposed aromatic cores, no additional advantage could be determined, while sulfur oxidation of the central sulfide proved detrimental.

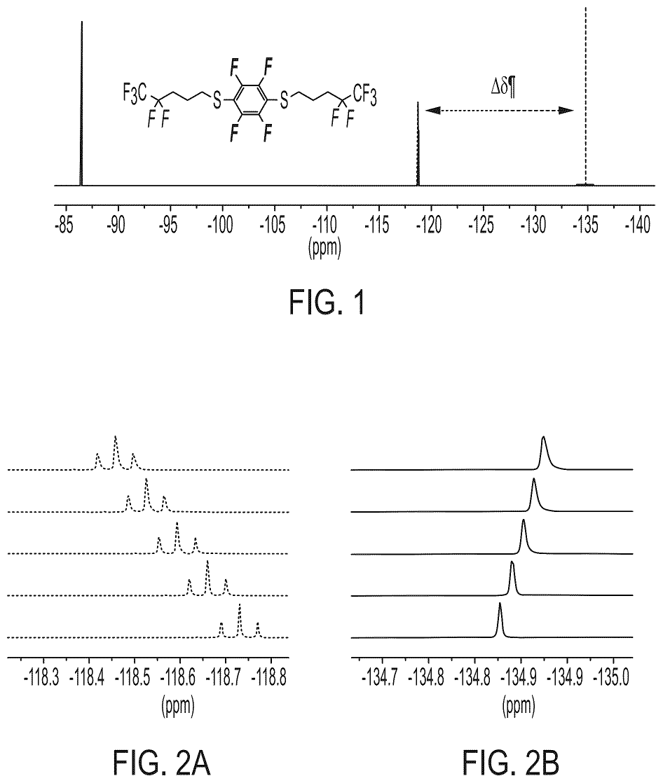

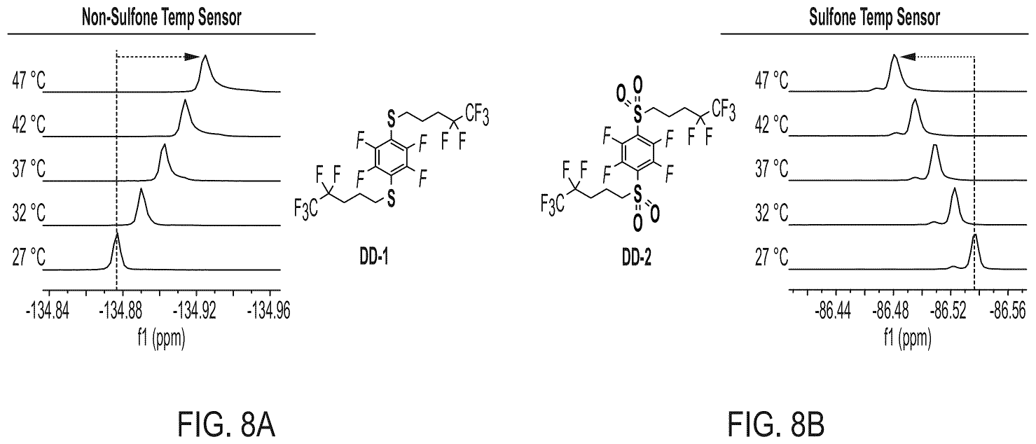

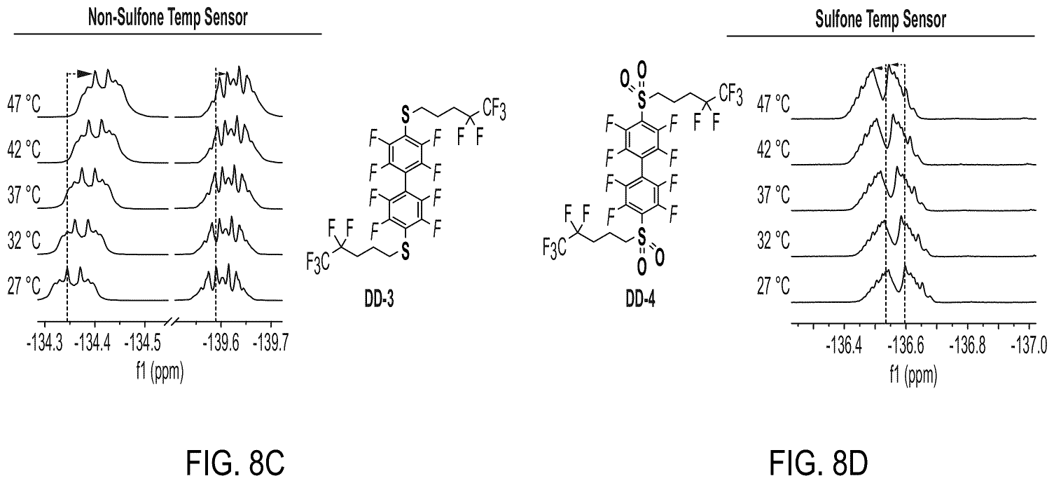

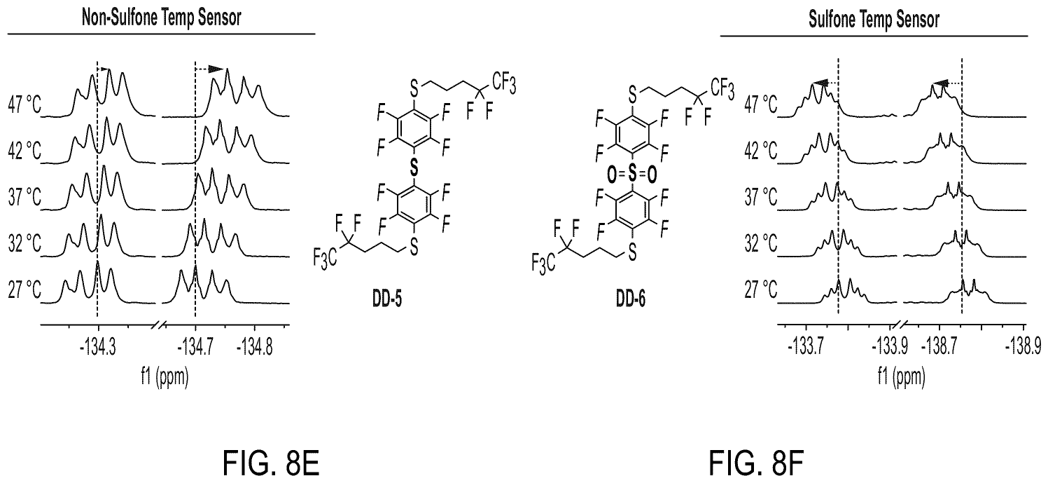

Surprisingly, a further look at the sulfonyl group-containing sensors revealed that the sulfonyl group whether within the core or substituted on the outer para positions of the aromatic core were also detrimental to the temperature response. In this case, DD-2 and DD-4, the oxidized analogs of the highly responsive DD-1 and DD-3, each lost 30% and 22% responsiveness from oxidation. The reduced responsiveness occurs based on a change in the direction of the chemical shift of the aryl fluorine in response to temperature, which now moves upfield (FIGS. 8A-F), whereas the resonance moves downfield in the case of DD-1 and DD-3 (FIG. 1) providing a larger chemical shift change relative to the CF₂ resonance.

FIG. 8A shows the temperature response of aryl ¹⁹F resonances in the spectra (aromatic region) of the non-sulfone containing temperature sensor DD-1 where aromatic resonance(s) move upfield with the increase of temperature.

FIG. 8B shows the temperature response of aryl ¹⁹F resonances in the spectra (aromatic region) of the sulfone containing temperature sensor DD-2 where aromatic resonance(s) move downfield with the increase of temperature.

FIG. 8C shows the temperature response of aryl ¹⁹F resonances in the spectra (aromatic region) of the non-sulfone containing temperature sensor DD-3 where aromatic resonance(s) move upfield with the increase of temperature.

FIG. 8D shows the temperature response of aryl ¹⁹F resonances in the spectra (aromatic region) of the sulfone containing temperature sensor DD-4 where aromatic resonance(s) move downfield with the increase of temperature.

FIG. 8E shows the temperature response of aryl ¹⁹F resonances in the spectra (aromatic region) of the non-sulfone containing temperature sensor DD-5 where aromatic resonance(s) move upfield with the increase of temperature.

FIG. 8F shows the temperature response of aryl ¹⁹F resonances in the spectra (aromatic region) of the sulfone containing temperature sensor DD-6 where aromatic resonance(s) move downfield with the increase of temperature.

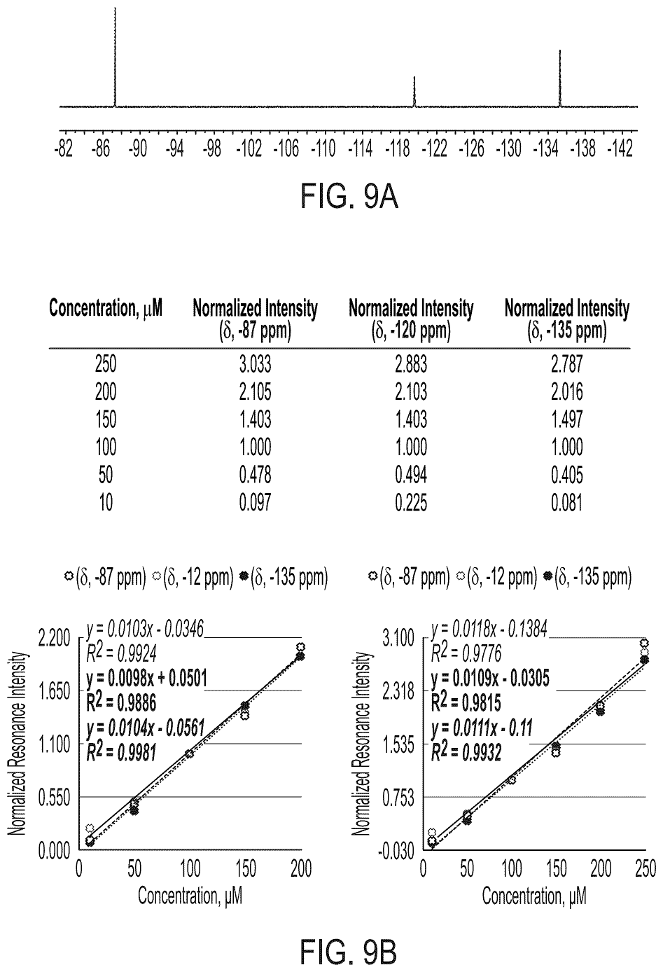

In addition to DD-1 and DD-3 having the desired liquid phase characteristics and being the most responsive sensors, these two sensor compounds were also shown to have sufficient aqueous solubility up to 200 μM (FIG. 9A) for analysis of their responsiveness in water. FIG. 9A shows the ¹⁹F NMR spectrum of DD-1 in phosphate buffer pH=6.9. In this case, PFTBA showed an aqueous responsiveness of 9.62×10⁻³ ppm/° C., similar to the literature value. DD-1 and DD-3 showed an 82% and 72% improvement in responsiveness with 17.57×10⁻³ ppm/° C. and 16.16×10⁻³ ppm/° C. respectively (FIG. 9B). FIG. 9B shows a table of normalized signal intensity of individual resonance at different concentrations of DD-1 (top). All signal intensities of resonances of 100 μM DD-1 were set to 1. At the bottom left is a normalized intensity chart of 3 individual resonances at different concentrations of DD-1, excluding 250 μM. At the bottom right is a normalized intensity chart of 3 individual resonances at different concentrations of DD-1, including 250 μM (micromolar). The R² of the CF₃ (−87 ppm) resonance line is only 0.9776.

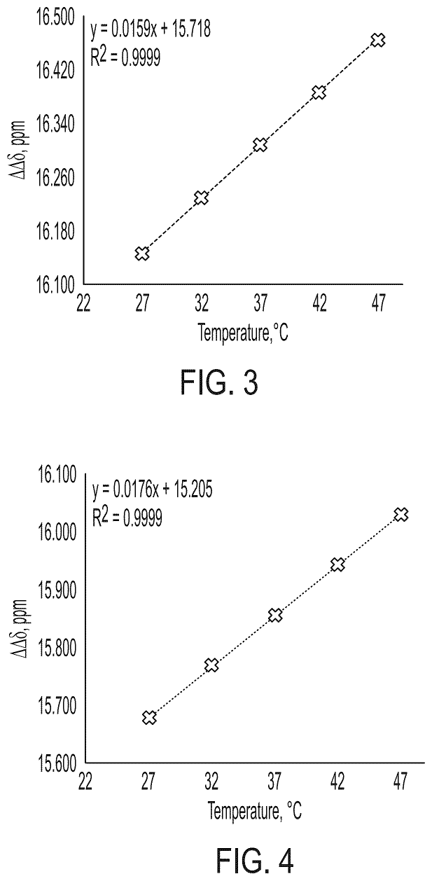

DD-1, DD-3, and DD-5 were all more responsive than the comparator compounds CC-1 PFTBA, CC-2 and CC-3. FIG. 1 shows the structure and spectrum of DD-1 in THF-d₈ at 27° C. Fluorine groups matched the 2 resonances determining the temperature responsiveness of DD-1. FIG. 2A shows the selected regions of the ¹⁹F NMR spectra of CF₂ groups of DD-1. Resonances move downfield as temperature increases from 27 to 47° C. FIG. 2B shows the selected regions of the ¹⁹F NMR spectra of Ar—F group of DD-1. Resonances move upfield as temperature increases from 27 to 47° C. FIG. 3 shows the temperature response of DD-1 in THF-d₈ with 15.98±0.05×10⁻³ ppm ° C.-1 (n=3, mean±SD). FIG. 4 shows the temperature response of DD-1 in phosphate buffer with 17.57±0.05×10⁻³ ppm ° C.-1 (n=3, mean±SD). In addition, DD-1 and DD-3 with short fluorinated alkyl tails (—CH₂CH₂CH₂CF₂CF₃) form a favorable liquid phase at room temperature for encapsulation with nanoparticles for ¹⁹F MRI experiments. (Table 1a), while a central sulfide in the aromatic core prevents room temperature liquid phase formation. Given its aqueous solubility, improved responsiveness could be measured for DD-1 in water, and predicts biological applications with this compound.

Degradation Studies of Temperature Sensors DD-1, DD-3, and DD-5 by Direct Photolysis and Ozonation:

The difference in degradation propensity between fluorinated functional groups when exposed to simulated environmental photolysis or oxidative water treatment conditions was investigated. Given concerns over the environmental persistence of highly fluorinated molecules, the degradation profiles of DD-1, DD-3, and DD-5 were evaluated under photolysis and oxidative conditions. In the case of oxidative conditions, Ozone, O₃ was chosen to simulate oxidation processes used in water treatment plants. Ozone is a selective oxidant that rapidly degrades in aqueous environments to an array of reactive oxygen species, including hydroxyl radical (·OH), a non-selective oxidant.

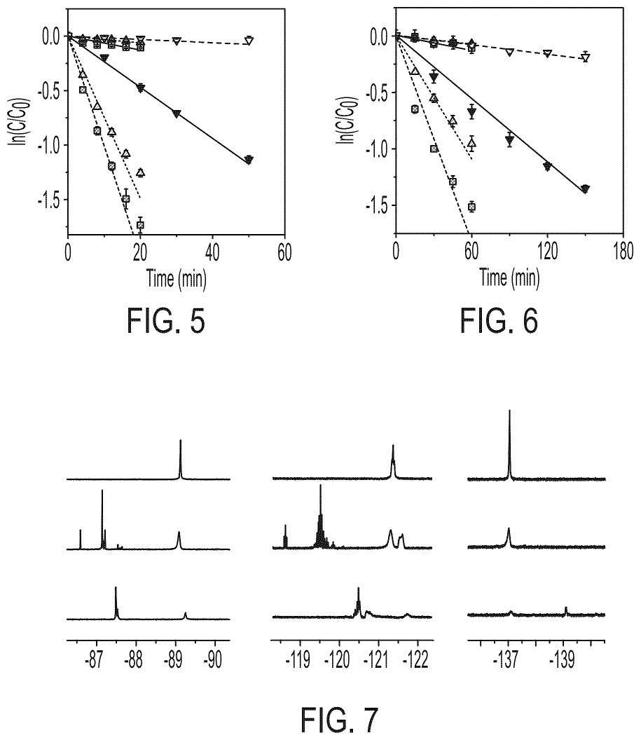

As a first experiment, UV-Vis absorption spectra of DD-1, DD-3, and DD-5 were obtained to evaluate the propensity to absorb sufficient light with wavelengths >290 nm under ambient conditions. UV-visible spectra show substantial overlap with the lamp output, indicating the potential for direct photolysis by sunlight or UV-lamps. Under both light sources DD-5 had the fastest rate constants followed by DD-3 then DD-1 (FIGS. 5 and 6).

The quantum yields, determined using the solar simulator, were 0.0272 mol Ei⁻¹ for DD-1, 0.0267 mol Ei⁻¹ for DD-3, and 0.0520 mol Ei⁻¹ for DD-5, with the higher quantum yields being consistent with the faster rate of degradation. For all compounds the kinetic rate constants in the mercury vapor lamp were larger compared to the solar simulator due to the high intensity UV light produced compared to the solar simulator which has a spectral energy distribution more similar to the sun. All compounds had half lives in the range of 7 to 30 minutes with the mercury vapor lamp and 25 to 72 minutes in the solar simulator.

### Fluorine Mass Balance Under Simulated Degradation Conditions:

Quantitative ¹⁹F NMR was conducted to determine if any of the fluorinated functional groups were susceptible to degradation. The environmental persistence and potential impact of these fluorinated temperature sensors will be a function of not only the persistence of the parent compound, but also that of any fluorinated reaction products. Photolysis and ozonation represent natural and engineered processes, respectively, that could degrade these compounds after their use and introduction into the water system.

¹⁹F NMR spectra were taken to quantify and partition degradation products from the parent compound signals for photolysis and ozonation (FIG. 7). Table 1a shows the mass balance for DD-1 determined using ¹⁹F-NMR under photolysis and ozonation conditions. The solutions analyzed contained 50 μM of DD-1 with 14 fluorine atoms, thus a total fluorine concentration of 700 μM, with 200 μM attributed to the aromatic fluorines (Ar—F; −137.04 ppm), 200 μM attributed to the CF₂ (−121.34 ppm), and 300 μM attributed to the CF₃ (−89.14 ppm) as shown in FIG. 7. FIG. 7 shows ¹⁹F-NMR spectra of DD-1 showing chemical shifts of fluorinated groups in the parent compound and reaction products. Top: regional spectrum of untreated DD-1. Middle: regional spectrum of DD-1 degraded by solar simulator. Bottom: regional spectrum of DD-1 degraded by ozone. (left) Spectra of CF₃ region. (middle) Spectra of CF₂ region. (right) Spectra of Ar—F region. For photolysis and ozonation, 61% and 87% the parent Ar—F signal was lost respectively. A peak at ˜139.1 ppm appeared following ozonation and is hypothesized to be a hydroxylated aromatic ring product, which is a known reaction process for aromatics during ozonation (Vialaton, D., et al (1998) J Photochem Photobiol A Chem 119 (1):39-45). For the parent CF₂, the signal decreased by 45% and 79%, but new CF₂ products appeared upfield. Under both degradative conditions, new peaks appear in the near regions of the CF₂, likely due to ring opening processes. These triplets and multiplets were allocated as CF₂ due to the splitting patterns and chemical shifts resembling those of CF₂ groups, and an assumption is that the CF₂ motif does not degrade. For the parent CF₃, the signal decreased by 38% and 69%, again with new CF₃ products appearing upfield. An excess of 39 μM and 48 μM fluorine for the solar simulator and ozonation, respectively, was found within the CF₃ region. These groups are hypothesized to be products produced by opening of the aromatic ring, which could be vinyl fluorines with oxygen and/or sulfur substituents.

During photolysis or ozonation of halogenated phenols, vinyl ring opening products are possible via oxidative free radical attack. Fluoride (F⁻) production via photolysis was 79 μM and 117 μM for ozonation. The F⁻ peak for ozonation is shifted slightly upfield compared to that of photolysis, but it was deemed the F⁻ peak because of the broad signal commonly seen experimentally (Bhat, A. P., et al (2022) Environ Sci Technol 56 (17):12336-12346). While fluoride production is believed to be from the aromatic fluorines, not all the Ar—F can be accounted for via F⁻. This is supported by the sum of the concentrations of fluorine for the Ar—F, F⁻, and ring opening products/vinyl F being 198 μM and 200 μM for photolysis and O₃, respectively. Given ˜10% error in quantification, these concentrations are equal to the initial Ar—F present. Similar trends were observed for DD-3 and DD-5 in the CF₂ and CF₃ region (SIX), with new peaks appearing near the parent CF₂ and CF₃ peaks.

FIG. 5 shows photochemical degradation kinetic plots of DD-1, DD-3, and DD-5 at 10 μM with photolysis (filled) and dark controls (empty) in (A) the mercury vapor lamp. Experiments were in 10 mM pH 7 phosphate buffer and contained residual DMSO from the stock solution. First order rate constants and 95% confidence intervals shown for each compound. Error bars indicate the standard deviation of three samples. Inverted triangles refer to DD-1, regular triangle refers to DD-3, and the squares refer to DD-5.

FIG. 6 shows photochemical degradation kinetic plots of DD-1, DD-3, and DD-5 at 10 μM with photolysis (filled) and dark controls (empty) in the solar simulator. Experiments were in 10 mM pH 7 phosphate buffer and contained residual DMSO from the stock solution. First order rate constants and 95% confidence intervals shown for each compound. Error bars indicate the standard deviation of three samples. Inverted triangles refer to DD-1, regular triangle refers to DD-3, and the squares refer to DD-5.

A difference between DD-1 and DD-3/DD-5 is that for the latter, the F⁻ peak is overlapping with the parent CF₂ peaks, thus F⁻ was calculated assuming that the CF₂ motif does not degrade. These degradation studies show the sensor compounds of Table 1a have a sufficient UV-visible absorption cross-section to enable aromatic photolysis degradation pathways which produce F⁻ and ring opening products. While the aromatic cores were found to undergo chemical transformations, these results show that the CF₂ and CF₃ motifs remain unchanged under these degradative conditions. Photolysis and H₂O₂ treatment have been shown to have limited capacity to degrade CF₂ and CF₃ alkyl motifs (Taniyasu, S., et al (2013) Chemosphere 90(5):1686-1692; Houtz, E. F., et al (2012) Environ Sci Technol 46(17):9342-9349; Hori, H., et al (2004) Environ Sci Technol 38 (22):6118-6124; Liu, F., et al (2022) J Hazard Mater 439:129580) but other processes under alkaline, high temperature conditions degrade these moieties (Hao, S., et al (2021) Environ Sci Technol 55 (5):3283-3295; Bentel, M. J., et al (2019) Environ Sci Technol 53 (7):3718-3728). Use of alkaline, high temperature treatment would require capture of excreted temperature sensors.

To address these challenges, the sensors DD-1 to DD-6 of Table 1a were designed with shorter fluorinated tails based on computational predictions, and to allow for evaluating heteroatom effects, and the aromatic core on phase behavior and temperature responsiveness.

The potential for degradation under simulated environmental and wastewater treatment conditions was explored for the Table 1a sensors. The results showed that the short fluorine chains, attached to a perfluoroaromatic core provide simplified and more responsive temperature sensors than comparators CC-1 PFTBA, CC-2 and CC-3, as well as a liquid phase for loading into nanoparticles. Degradation analyses further show that incorporation of arylfluorine groups lead to more readily degraded compounds than perfluoroalkyl substances (PFAS) and may be beneficial for designing additional organofluorine compounds and materials beyond temperature sensors.

### Nanoparticle Encapsulation:

Nanoparticles with encapsulated small molecules have been investigated for various biomedical applications (Koshkina O, et al (2019) Adv Funct Mater. May 9; 29(19):1806485; Nakamura, T.; et al (2015) Chem. Sci. 2015, 6, 1986-1990; Nakamura, T., et al (2015) Angew. Chem., Int. Ed., 54:1007-1010; Pochert, A., et al (2017) J. Colloid Interface Sci. 488:1-9). Liquid small molecules are capable of being entrapped inside hollow nanoparticles via a postsynthetic loading method (Lee, A. L., et al (2017) ACS Nano, 11(6):5623-5632; Lee, A. L., et al (2021) Langmuir 37(17):5222-5231). Liquid Formula I sensor compounds may be loaded into silica nanoparticles and extracted into aqueous phases while varying the sonication time, concentration of nanoparticles, volume ratio between aqueous and fluorous phases, and pH of the extraction water. Loading yields and efficiency are determined via ¹⁹F nuclear magnetic resonance and N₂ physisorption isotherms. Sonication time may have a significant correlation to loading yield and efficiency.

Metal-oxide or polymeric nanoparticles act as stable carrier agents for fluorous material and organofluorine liquids (Koshkina, O., et al. (2020) J. Colloid Interface Sci. 565:278-287). Mesoporous silica nanoparticles are biocompatible and have tunable surfaces to incorporate colloidal stabilizers, targeting agents, fluorescent dyes, and therapeutic drugs (Pednekar, P. P., et al, Chapter 23—Mesoporous silica nanoparticles: a promising multifunctional drug delivery system. In Nanostructuresfor Cancer Therapy; Ficai, A., Grumezescu, A. M., Eds.; Elsevier: Micro and Nano Technologies, (2017); pp 593-621).

Nanoparticles have been generated by forming an organofluorine emulsion where the silica precursors are templated onto the surface of the emulsion bubble (Matsushita, H. Multifunctional 19F MRI Contrast Agents Based on Core-Shell Fluorine-Encapsulated Silica Nanoparticles; (2014) Osaka University). Since the organofluorine liquids are trapped inside solid cages, they are resistant to coalescence and Ostwald ripening as the solid cages are unlikely to merge into one particle or to grow in size as a function of PFC volume. Ultraporous mesostructured silica nanoparticles (UMNs) loaded with various organofluorine liquids via a postsynthetic loading method (PSLM) have been reported encapsulating different perfluorocarbons (Lee, A. L., et al (2017) ACS Nano. 11(6):5623-5632; Matsushita, H.; et al. (2014) Angew. Chem., Int. Ed. 53:1008-1011).

A rational design of highly temperature responsive fluorinated small compounds is demonstrated for magnetic resonance-based thermometry. Compounds of Table 1a are more sensitive than CC-1 PFTBA which has limited water solubility and is a solid at room temperature limiting facile use in aqueous systems for loading into nanoparticles. Additionally, the high fluorinated alkyl tail of CC-1 PFTBA raises concerns over environmental persistence. Exemplary Table 1a compound DD-1 provides further improvement in temperature responsiveness, while greatly reducing the non-magnetically equivalent fluorine atoms to maintain high signal sensitivity while minimizing chemical shift artifacts. Based on a combined analytical approach of quantitative ¹⁹F NMR and HPLC analysis, the advantages of using arylfluorine groups in organofluorine designs are demonstrated, which provide a mechanism for sufficient absorption of UV light to enhance environmental photolytic degradation as well as degradation under water processing conditions relative to more persistent aliphatic fluorine groups. The Formula I sensor compounds may also find use in combination with magnetic resonance-based oximetry measurements which are temperature sensitive, as well as nanoparticle encapsulation for MRI-based thermometry applications.

### EXAMPLES

### Synthesis of DD Compounds:

DD compounds were synthesized as described in published protocols with modifications and following the General Scheme:

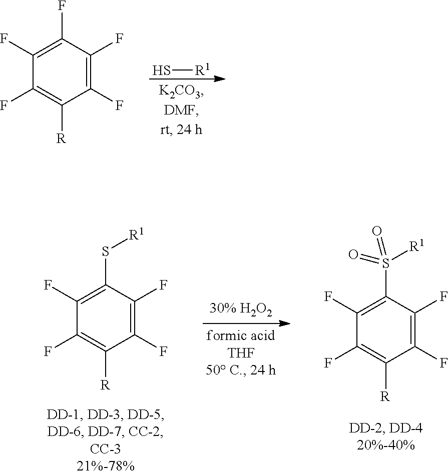

Materials: Hexafluorobenzene was purchased from Oakwood Chemical (Estill, SC). Decafluorobiphenyl was purchased from Sigma-Aldrich (St. Louis, MO). Pentafluorophenyl sulfide was purchased from Matrix Scientific (Columbia, SC). 4,4,5,5,5-Pentafluoro-1-pentanethiol was purchased from Ambeed (Arlington Heights, IL). 3,3,4,4,5,5,6,6,7,7,8,8,9,9,10,10,10-heptadecafluorodecane-1-thiol was purchased from Santa Cruz Biotechnology (Dallas, TX). Other chemicals and reagents were purchased from Sigma-Aldrich (St. Louis, MO) without further purification.

### Preparation ofpentafluorophenyl sulfone, 6,6′-sulfonylbis(1,2,3,4,5-pentafluorobenzene)

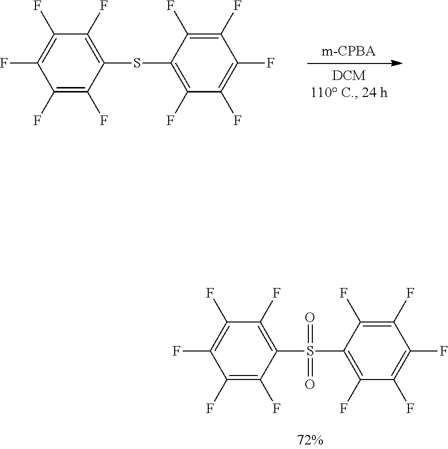 [SMILES: Fc1c(F)c(F)c(Sc2c(F)c(F)c(F)c(F)c2F)c(F)c1F ; O=S(=O)(c1c(F)c(F)c(F)c(F)c1F)c1c(F)c(F)c(F)c(F)c1F]

Pentafluorophenyl sulfide, bis(perfluorophenyl)sulfane (915 mg, 2.50 mmol) and meta-chloroperbenzoic acid, m-CPBA (1726 mg, 10.00 mmol) were suspended in 10 mL of dichloromethane, DCM in a seal tube. The reaction mixture was heated in a 110° C. oil bath for 24 h. Reaction mixture was then cooled to room temperature and extra DCM was added to dissolve all the precipitates. Reaction mixture was washed with 1M K₂CO₃ (50 mL×3). Organic layer was then washed with brine, dried with anhydrous MgSO₄, and concentrated under reduced pressure. The crude product was purified by Combi-flash chromatography (5% ethyl acetate in hexane) to obtain pentafluorophenyl sulfone as white solid (717 mg, 72%). ¹³C NMR (126 MHz, Chloroform-d) δ 146.00, 145.47, 138.22, 116.70. ¹⁹F NMR (471 MHz, Chloroform-d) δ −135.25 (m, J=19.1, 9.9, 9.1, 5.9 Hz), −140.54 (m, J=21.3, 8.1 Hz), −157.02 (m, J=20.9, 7.0 Hz).

### Preparation of perfluoro-1,4-phenylene)bis((4,4,5,5,5-pentafluoropentyl)sulfane, DD-1

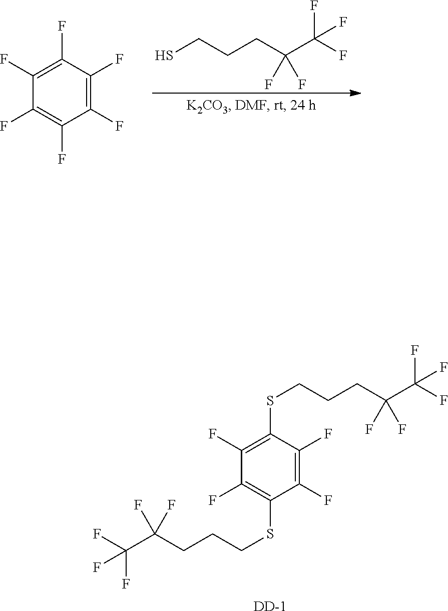 [SMILES: Fc1c(F)c(F)c(F)c(F)c1F ; FC(F)(F)C(F)(F)CCCS ; Fc1c(F)c(SCCCC(F)(F)C(F)(F)F)c(F)c(F)c1SCCCC(F)(F)C(F)(F)F ; "DD-1"]

Hexafluorobenzene (23 μL, 0.20 mmol) was dissolved in 3 mL of DMF followed by addition of 4,4,5,5,5-pentafluoro-1-pentanethiol (60 μL, 0.40 mmol) and then potassium carbonate, K₂CO₃ (80 mg, 0.60 mmol) to the solution. The resulting reaction mixture was stirred at room temperature for 24 h. The reaction was quenched with brine (10 mL) and extracted with ethyl acetate (5 mL×3). The organic layers were combined, dried with anhydrous MgSO₄, and concentrated under reduced pressure. The crude product was purified by Combi-flash chromatography (0-100% ethyl acetate in hexane) to obtain DD-1 as clear oil. (78 mg, 74%). ¹H NMR (500 MHz, Chloroform-d) δ 3.02 (t, J=7.0 Hz, 1H), 2.23 (tt, J=17.8, 7.9 Hz, 2H), 1.86 (dt, J=14.7, 7.1 Hz, 1H). ¹³C NMR (126 MHz, THF) δ 147.38, 119.41, 116.07, 114.23, 33.56, 28.75, 20.94. ¹⁹F NMR (471 MHz, Chloroform-d) δ −85.49, −118.01 (t, J=18.1 Hz), −133.09.

### Preparation of 1,2,4,5-tetrafluoro-3,6-bis((4,4,5,5,5-pentafluoropentyl)sulfonyl)benzene, DD-2

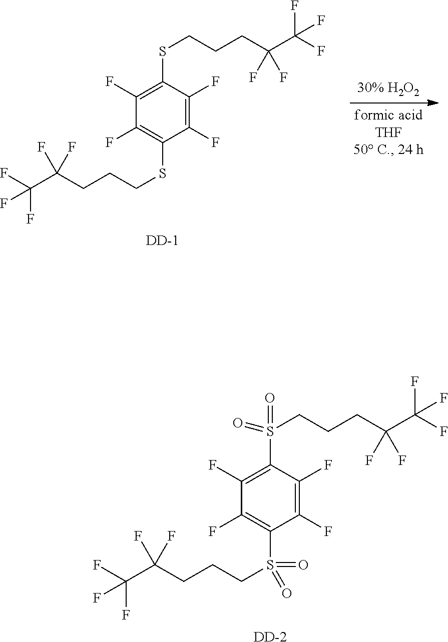 [SMILES: Fc1c(F)c(SCCCC(F)(F)C(F)(F)F)c(F)c(F)c1SCCCC(F)(F)C(F)(F)F ; "DD-1" ; O=S(=O)(CCCC(F)(F)C(F)(F)F)c1c(F)c(F)c(S(=O)(=O)CCCC(F)(F)C(F)(F)F)c(F)c1F ; "DD-2"]

Perfluoro-1,4-phenylene)bis((4,4,5,5,5-pentafluoropentyl)sulfane, DD-1 (50 mg, 0.094 mmol) was dissolved in 2 mL of THF. Formic acid (600 μL, 15.90 mmol) and 30% hydrogen peroxide (0.75 g, 6.62 mmol) were added to the solution. The resulting reaction mixture was stirred at 50° C. for 24 h. The reaction was quenched with brine (10 mL) and extracted with ethyl acetate (5 mL×3). The organic layers were combined, dried with anhydrous MgSO₄, and concentrated under reduced pressure. The crude product was purified by Combi-flash chromatography (0-100% ethyl acetate in hexane) to obtain DD-2 as white solid. (10 mg, 18%). ¹H NMR (500 MHz, THF-d₈) δ 3.68-3.61 (m, 1H), 2.40 (tt, J=18.2, 8.2 Hz, 2H), 2.13 (p, J=7.8 Hz, 1H). ¹³C NMR (126 MHz, THF) δ 145.31, 123.45, 119.31, 115.78, 55.87, 28.56, 14.15. ¹⁹F NMR (471 MHz, THF-d₈) δ −86.54, −119.34 (t, J=18.6 Hz), −135.49.

### Preparation of perfluoro-[1,1′-biphenyl]-4,4′-diyl)bis((4,4,5,5,5-pentafluoropentyl)sulfane (DD-3)

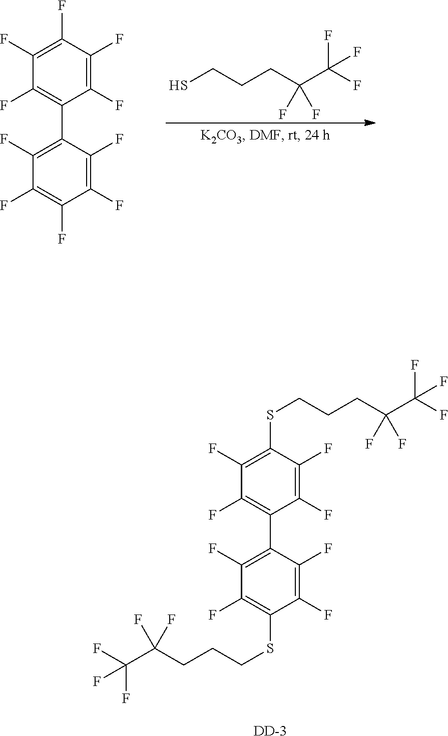 [SMILES: Fc1c(F)c(F)c(-c2c(F)c(F)c(F)c(F)c2F)c(F)c1F ; FC(F)(F)C(F)(F)CCCS ; Fc1c(F)c(-c2c(F)c(F)c(SCCCC(F)(F)C(F)(F)F)c(F)c2F)c(F)c(F)c1SCCCC(F)(F)C(F)(F)F ; "DD-3"]

Decafluorobiphenyl, perfluoro-1,1′-biphenyl (68 mg, 0.20 mmol) was dissolved in 3 mL of dimethylformamide, DMF. 4,4,5,5,5-Pentafluoro-1-pentanethiol (60 μL, 0.40 mmol) was added to the solution followed by addition of K₂CO₃ (80 mg, 0.60 mmol). The resulting reaction mixture was stirred at room temperature for 24 h. The reaction was quenched with brine (10 mL) and extracted with ethyl acetate (5 mL×3). The organic layers were combined, dried with anhydrous MgSO₄, and concentrated under reduced pressure. The crude product was purified by Combi-flash chromatography (0-100% ethyl acetate in hexane) to obtain DD-3 as clear oil. (84 mg, 62%). ¹H NMR (500 MHz, Chloroform-d) δ 3.11 (t, J=7.0 Hz, 1H), 2.26 (tt, J=17.8, 7.8 Hz, 2H), 1.94 (p, J=7.2 Hz, 1H). ¹³C NMR (126 MHz, THF) δ 147.44, 144.22, 119.42, 117.05, 116.08, 106.78, 33.55, 28.82, 21.15. ¹⁹F NMR (471 MHz, Chloroform-d) δ −85.46, −117.95 (t, J=17.8 Hz), −132.79-−132.93 (m), −137.29 (dq, J=19.9, 7.0 Hz).

### Preparation of 2,2′,3,3′,5,5′,6,6′-octafluoro-4,4′-bis((4,4,5,5,5-pentafluoropentyl)sulfonyl)-1,1′-biphenyl, DD-4

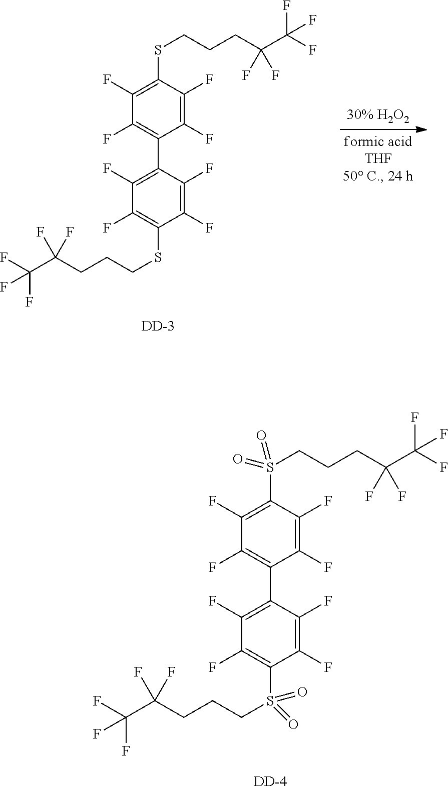 [SMILES: Fc1c(F)c(-c2c(F)c(F)c(SCCCC(F)(F)C(F)(F)F)c(F)c2F)c(F)c(F)c1SCCCC(F)(F)C(F)(F)F ; "DD-3" ; O=S(=O)(CCCC(F)(F)C(F)(F)F)c1c(F)c(F)c(-c2c(F)c(F)c(S(=O)(=O)CCCC(F)(F)C(F)(F)F)c(F)c2F)c(F)c1F ; "DD-4"]

DD-3 (50 mg, 0.073 mmol) was dissolved in 2 mL of THF. Formic acid (600 μL, 15.90 mmol) and 30% hydrogen peroxide (0.75 g, 6.62 mmol) were subsequently added to the solution. The resulting reaction mixture was stirred at 50° C. for 24 h. The reaction was quenched with brine (10 mL) and extracted with ethyl acetate (5 mL×3). The organic layers were combined, dried with anhydrous MgSO₄, and concentrated under reduced pressure. The crude product was purified by Combi-flash chromatography (0-100% ethyl acetate in hexane) to obtain DD-4 as white solid. (19 mg, 35%). ¹H NMR (500 MHz, THF-d₈) δ 3.74-3.67 (m, 4H), 2.45 (td, J=18.5, 9.9 Hz, 4H), 2.21 (p, J=7.8 Hz, 4H). ¹³C NMR (126 MHz, THF) δ 145.19, 144.57, 121.68, 119.33, 115.83, 111.20, 55.95, 28.61, 14.16. ¹⁹F NMR (471 MHz, THF-d₈) δ −86.51, −119.30 (t, J=18.6 Hz), −136.43-−136.72 (m).

### Preparation of bis(2,3,5,6-tetrafluoro-4-((4,4,5,5,5-pentafluoropentyl)thio)phenyl)sulfane, DD-5

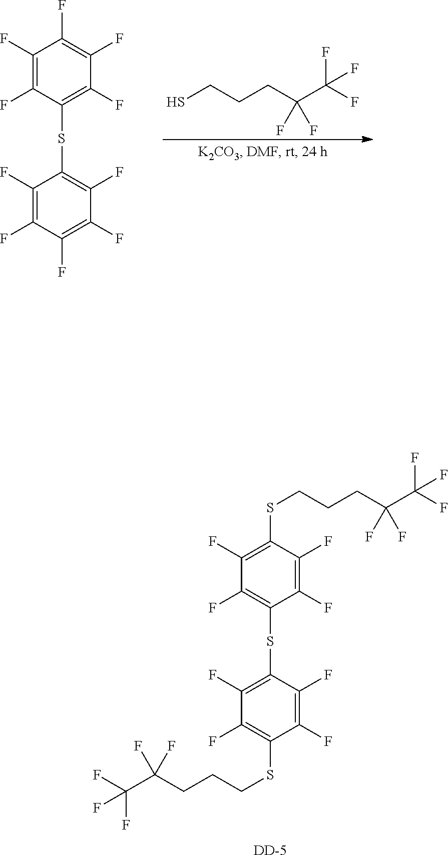 [SMILES: Fc1c(F)c(F)c(Sc2c(F)c(F)c(F)c(F)c2F)c(F)c1F ; FC(F)(F)C(F)(F)CCCS ; Fc1c(F)c(Sc2c(F)c(F)c(SCCCC(F)(F)C(F)(F)F)c(F)c2F)c(F)c(F)c1SCCCC(F)(F)C(F)(F)F ; "DD-5"]

Bis(perfluorophenyl)sulfane (72 mg, 0.20 mmol) was dissolved in 3 mL of DMF. 4,4,5,5,5-Pentafluoro-1-pentanethiol (60 μL, 0.40 mmol) was subsequently added to the solution followed by K₂CO₃ (80 mg, 0.60 mmol). The resulting reaction mixture was stirred at room temperature for 24 h. The reaction was quenched with brine (10 mL) and extracted with ethyl acetate (5 mL×3). The organic layers were combined, dried with anhydrous MgSO₄, and concentrated under reduced pressure. The crude product was purified by Combi-flash chromatography (0-100% ethyl acetate in hexane) to obtain DD-5 as white solid. (112 mg, 78%). ¹H NMR (500 MHz, THF-d₈) δ 3.10 (t, J=7.1 Hz, 4H), 2.31 (tt, J=18.4, 8.0 Hz, 4H), 1.85 (p, J=7.3 Hz, 4H). ¹³C NMR (126 MHz, THF) δ 147.25, 146.97, 119.39, 116.41, 116.04, 111.25, 33.45, 28.77, 21.03. ¹⁹F NMR (471 MHz, THF-d₈) δ −86.50, −118.76 (t, J=18.7 Hz), −134.28 (dd, J=25.1, 11.0 Hz), −134.71 (dd, J=24.8, 11.4 Hz).

### Preparation of sulfonylbis(2,3,5,6-tetrafluoro-4,1-phenylene))bis((4,4,5,5,5-pentafluoropentyl)sulfane (DD-6)

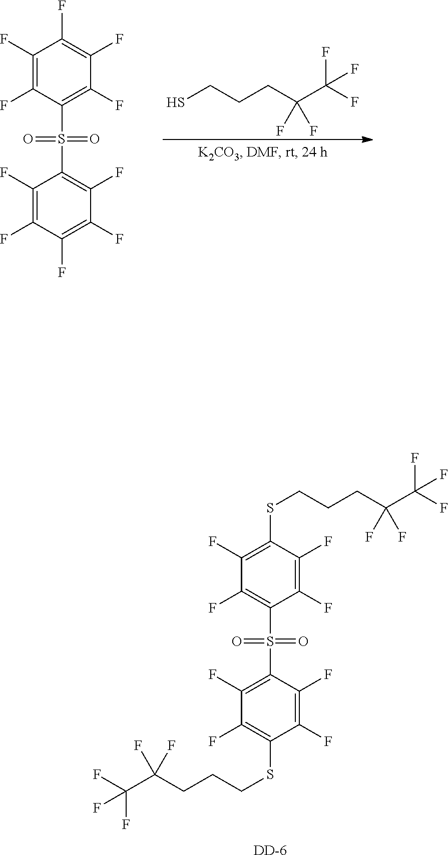 [SMILES: O=S(=O)(c1c(F)c(F)c(F)c(F)c1F)c1c(F)c(F)c(F)c(F)c1F ; FC(F)(F)C(F)(F)CCCS ; O=S(=O)(c1c(F)c(F)c(SCCCC(F)(F)C(F)(F)F)c(F)c1F)c1c(F)c(F)c(SCCCC(F)(F)C(F)(F)F)c(F)c1F ; "DD-6"]

Pentafluorophenyl sulfone, 6,6′-sulfonylbis(1,2,3,4,5-pentafluorobenzene) (80 mg, 0.20 mmol) was dissolved in 3 mL of DMF. 4,4,5,5,5-Pentafluoro-1-pentanethiol (60 μL, 0.40 mmol) was subsequently added to the solution followed by K₂CO₃ (80 mg, 0.60 mmol). The resulting reaction mixture was stirred at room temperature for 24 h. The reaction was quenched with brine (10 mL) and extracted with ethyl acetate (5 mL×3). The organic layers were combined, dried with anhydrous MgSO₄, and concentrated under reduced pressure. The crude product was purified by Combi-flash chromatography (0-100% ethyl acetate in hexane) to obtain DD-6 as white solid. (79 mg, 53%). ¹H NMR (500 MHz, Chloroform-d) δ 3.18 (t, J=7.2 Hz, 4H), 2.21 (tt, J=17.6, 7.7 Hz, 4H), 1.94 (p, J=7.3 Hz, 4H). ¹³C NMR (126 MHz, THF) δ 146.92, 144.54, 123.45, 119.38, 119.32, 116.01, 33.02, 28.83, 21.27. ¹⁹F NMR (471 MHz, Chloroform-d) δ −85.47, −117.98 (t, J=18.0 Hz), −131.49 (td, J=16.0, 7.6 Hz), −136.45-−136.57 (m).

### Preparation of (sulfonylbis(2,3,5,6-tetrafluoro-4,1-phenylene))bis((3,3,4,4,5,5,6,6,7,7,8,8,9,9,10,10,10-heptadecafluorodecyl)sulfane, DD-7

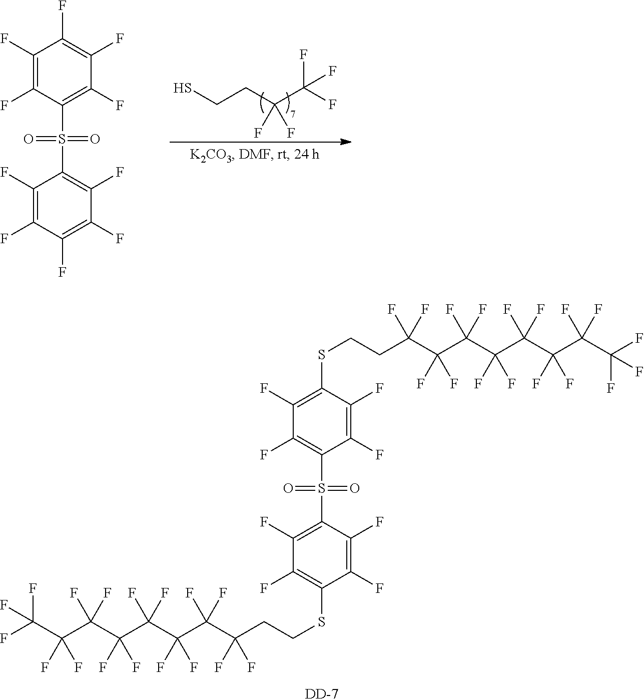

Pentafluorophenyl sulfone (80 mg, 0.20 mmol) was dissolved in 3 mL of DMF. 3,3,4,4,5,5,6,6,7,7,8,8,9,9,10,10,10-heptadecafluorodecane-1-thiol (114 μL, 0.40 mmol) was subsequently added to the solution followed by K₂CO₃ (80 mg, 0.60 mmol). The resulting reaction mixture was stirred at room temperature for 24 h. The reaction was quenched with brine (10 mL) and extracted with ethyl acetate (5 mL×3). The organic layers were combined, dried with anhydrous MgSO₄, and concentrated under reduced pressure. The crude product was purified by Combi-flash chromatography (0-100% ethyl acetate in hexane) to obtain DD-7 as white solid. (54 mg, 20%). ¹H NMR (500 MHz, THF-d₈) δ 3.50-3.39 (m, 1H), 2.68 (tt, J=18.0, 8.0 Hz, 1H). ¹³C NMR (126 MHz, THF) δ 146.83, 144.57, 122.98, 119.51, 117.95, 117.30, 111.34, 111.10, 111.02, 110.97, 110.45, 108.62, 31.77, 24.99. ¹⁹F NMR (471 MHz, THF-d₈) δ −81.76, −114.54, −121.96-−122.68 (m), −123.27, −123.82, −126.76, −133.75 (dt, J=15.5, 7.9 Hz), −138.60 (q, J=13.2, 12.4 Hz).

¹⁹F NMR Variable Temperature Measurements: For temperature measurements in organic solvent, compounds were dissolved in THF-d₈ and measured with Bruker AVANCE III 500 equipped with a 5 mm BBFO SmartProbe. For measure of compounds dissolved in THF-d₈, ¹⁹F spectra were obtained at 471 MHz with dummy scans=4, acquisition time=0.57 s, delay time=1 s, pre-scan delay=μs, and the number of scans=16. The temperature was increased by passing heated N₂ gas over the spinning sample which was monitored by an internal instrument temperature probe. The temperature was allowed to stabilize for 3 min before scanning. For temperature measurements in aqueous solution, compounds were dissolved in DMSO first and then added to 10 mM pH=6.9 phosphate buffer/D₂O (9:1 v/v) to make 5% DMSO stock in 500 μL phosphate buffer/D₂O. Samples were measured with Bruker AVANCE III 500@ equipped with a 5 mm BBFO SmartProbe®. ¹⁹F spectra were obtained at 471 MHz with dummy scans=4, acquisition time=0.57 s, delay time=1 s, pre-scan delay=μs, and the number of scans=2048. The temperature was increased by passing heated N₂ gas over the spinning sample which was monitored by an internal instrument temperature probe. The temperature was allowed to stabilize for 10 min before scanning.

¹⁹F NMR Aqueous Solubility Limit Measurements: Compound was dissolved in DMSO and then added to 10 mM pH=6.9 phosphate buffer/D₂O (9:1 v/v) to make 5% DMSO stock in 500 μL phosphate buffer/D₂O at concentration of 10/50/100/150/200/250 μM. Samples were measured with Bruker AVANCE III 500@ equipped with a 5 mm BBFO SmartProbe®. ¹⁹F spectra were obtained at 471 MHz with dummy scans=4, acquisition time=0.57 s, delay time =1 s, pre-scan delay=p s, and the number of scans=800 UV-Visible Spectra: Stock solutions of DD-1, DD-3, and DD-5 were prepared at 10 mM in DMSO and stored in the dark. A 10 mM phosphate buffer at pH=7 was prepared and stock solutions were diluted to 50 μM. UV-visible absorption spectra were obtained using a Horiba Aqualog®.

Photolysis Experiments: Stock solutions were diluted to 10 μM in the phosphate buffer for kinetic experiments and 50 μM for ¹⁹F NMR experiments. Solutions were placed in 10 mL quartz tubes sealed with cork stoppers with no contact to the solution. The solutions were photolyzed in (i) an Atlas Suntest CPS+® solar simulator with a 1500 W xenon lamp at an intensity of 765 W m-2 using a wavelength range of 290-800 nm at a 30° angle, and (ii) a 450 W medium pressure polychromatic mercury vapor lamp with a quartz immersion well with cooling water circulation, a Pyrex 280 nm cutoff filter (Ace Glass), and a merry-go-round sample holder. Samples were photolyzed until ˜60 to 80% degradation of the parent compound was achieved. For each compound an equivalent tube was prepared and wrapped in aluminum foil as a dark control. Experiments were performed in triplicate

Photolysis Kinetics and Quantum Yields: A minimum of five spaced time points ranging from 60-180 min for the solar simulator and 20-60 min for the mercury vapor lamp were taken to determine the loss of the parent compound concentration using high-pressure liquid chromatography (HPLC) combined with a variable wavelength UV detector (Agilent 1100 series). First order rate constants were found by regression of ln(C/Co) versus time where Co is the initial concentration and C is the concentration in μM. The direct photolysis quantum yields in the solar simulator were found using a p-nitroanisole-pyridine (PNA-PYR) actinometer and relationships (Dulin and Mill (1982) Environ. Sci. Technol. 16(11):815-820; Leifer, A. The Kinetics of Environmental Aquatic Photochemistry: Theory and Practice; American Chemical Society, 1988) and updated calculations (Laszakovits et al (2017) Environ Sci Technol Lett 4(1):11-14).

Ozone-mediated Degradation: The 10 mM DMSO stock solution of DD-1 was used to make 100 mL of a 50 μM solution in the 10 mM pH=7 phosphate buffer. The solution was placed in an Erlenmeyer flask in an ice bath. Ozone (O₃) was produced using ultra-high pure oxygen gas (99.99%) with a Pacific Ozone O₃ generator and bubbled directly into the sample solution. O₃ concentration was previously measured using UV-Vis spectroscopy (Beckman Coulter DU 530). The concentration of O₃ (with an ε=3000 M⁻¹ cm⁻¹) was determined by direct absorbance at 258 nm. By using a 3:1 dilution, the absorbance obtained is equivalent to the O₃ concentration in mM. After 30 minutes the O₃ concentration in ultrapure water was 0.61 mM or 29.16 mg/L. Samples were taken over time for 300 minutes and parent compound degradation was monitored via HPLC.

All publications, patents, and patent documents are incorporated by reference herein as though individually incorporated by reference. The invention has been described with reference to various specific and preferred embodiments and techniques. However, it should be understood that many variations and modifications may be made while remaining within the spirit and scope of the invention

What is claimed is:

1. A compound of Formula I: 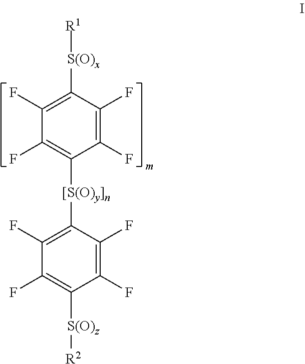
or a pharmaceutically acceptable salt, stereoisomer, mixture of stereoisomers, tautomer, or isotopic analog thereof, wherein:
R¹ and R² are independently selected from —(C₃-C₆ alkyldiyl)-(CF₂)ₚ—CF₃, where p is 1 to 10;
x, y, and z are independently selected from 0, 1, and 2; and
m and n are independently selected from 0 and 1, where if n is 1, then m is 1.

2. The compound of claim 1 wherein C₃-C₆ alkyldiyl is —CH₂CH₂CH₂—.

3. The compound of claim 1 wherein p is 1.

4. The compound of claim 1 wherein R¹ and R² are each —CH₂CH₂CH₂CF₂CF₃.

5. The compound of claim 1 wherein x, y, and z are each 0.

6. The compound of claim 1 wherein x is 0, y is 2, and z is 0.

7. The compound of claim 1 wherein m is 1 and n is 1.

8. The compound of claim 1 wherein m is 1 and n is 0.

9. The compound of claim 1 wherein m is 0 and n is 0.

10. A compound selected from the group consisting of:
(perfluoro-1,4-phenylene)bis((4,4,5,5,5-pentafluoropentyl)sulfane);
1,2,4,5-tetrafluoro-3,6-bis((4,4,5,5,5-pentafluoropentyl)sulfonyl)benzene;
(perfluoro-[1,1′-biphenyl]-4,4′-diyl)bis((4,4,5,5,5-pentafluoropentyl)sulfane);
2,2′,3,3′,5,5′,6,6′-octafluoro-4,4′-bis((4,4,5,5,5-pentafluoropentyl)sulfonyl)-1,1′-biphenyl;
bis(2,3,5,6-tetrafluoro-4-((4,4,5,5,5-pentafluoropentyl)thio)phenyl)sulfane;
(sulfonylbis(2,3,5,6-tetrafluoro-4,1-phenylene))bis((4,4,5,5,5-pentafluoropentyl)sulfane); and
(sulfonylbis(2,3,5,6-tetrafluoro-4,1-phenylene))bis((3,3,4,4,5,5,6,6,7,7,8,8,9,9,10,10,10-heptadecafluorodecyl)sulfane).

11. A nanoparticle formulation comprising a compound of Formula I of claim 1 and a nanoparticle.

12. The nanoparticle formulation of claim 11 wherein the nanoparticle is a mesoporous silica nanoparticle.

13. A method for measurement of temperature in a biological sample comprising detecting the Formula I compound of claim 1 in a biological sample by ¹⁹F magnetic resonance imaging at two or more temperatures.
